(19) United States
(12) Patent Application Publication
(10) Pub. No.: US 2024/0099592 A1
(43) Pub. Date: Mar. 28, 2024

(54) MONITORING OF BREATHING AND HEART FUNCTION

(71) Applicant: THE CHILDREN'S MEDICAL CENTER CORPORATION, Boston, MA (US)

(72) Inventors: Benjamin MATTHEWS, Newton, MA (US); Christopher NORVE, Boston, MA (US)

(73) Assignee: THE CHILDREN'S MEDICAL CENTER CORPORATION, Boston, MA (US)

(21) Appl. No.: 17/769,052

(22) PCT Filed: Oct. 8, 2020
(86) PCT No.: PCT/US2020/054747

§ 371 Date: Apr. 14, 2022

Related U.S. Application Data
(60) Provisional application No. 62/915,170, filed on Oct. 15, 2019.

(51) Int. Cl. A61B 5/0205; A61B 5/00; A61B 5/085; A61B 5/318

(52) U.S. Cl. CPC A61B 5/0205; A61B 5/085; A61B 5/318; A61B 5/6801; A61B 5/7239; A61B 5/7275; A61B 5/7282; A61B 5/742; A61B 5/0002; A61B 2560/045; A61B 2560/0462

(57) ABSTRACT

A system includes a sensor configured to generate thoracic impedance data associated with a patient for a period of time, a memory storing machine-readable instructions, and a control system communicatively coupled to the sensor. The control system is arranged to provide control signals to one or more electronic devices and including one or more processors configured to execute the machine-readable instructions to analyze the generated thoracic impedance data associated with the patient; determine, based at least in part on the analysis, respiratory function signals and cardiac function signals, and whether the respiratory function signals and cardiac function signals satisfies a predetermined threshold, and cause an operation of the one or more electronic devices to be modified in response to determining the respiratory function signals and cardiac function signals satisfies the predetermined threshold.

### CROSS-REFERENCE TO RELATED APPLICATIONS

This application claims benefit under 35 U.S.C. § 119(e) of U.S. Provisional Application No. 62/915,170 filed Oct. 15, 2019, the contents of which are incorporated herein by reference in their entirety.

### TECHNICAL FIELD

The present disclosure relates generally to systems and methods for non-invasive real time monitoring of respiratory and cardiac functions, and providing a notification to a user or a caregiver upon exceeding critical values.

### BACKGROUND

A large population of patients with chronic medical conditions (such as asthma, COPD, severe food allergies, history of anaphylaxis, heart failure, etc.) are at risk of acute disease exacerbation. Those patients who are at risk of acute disease exacerbation may experience potentially fatal medical emergencies. A significant proportion of the patients who succumb to acute disease exacerbation die before the arrival of emergency medical personnel.

The timing of when patients seek medical attention profoundly determines the outcome of the acute disease exacerbation. Untreated medical emergencies that arise in patients with chronic medical conditions and that culminate in death do so over a period of minutes to days. These chronic medical conditions are ultimately characterized in the end by respiratory and cardiac failure. Too often patients do not recognize or appreciate the severity of symptoms and, therefore, delay calling for help. Importantly, there are no existing non-invasive monitoring and disease severity scoring technologies capable of alerting patients of medical emergencies. An early warning signal instructing these patients to seek medical attention for advancing respiratory or cardiac distress prior to them becoming critically ill would be of monumental importance in preventing morbidity and mortality.

### SUMMARY

According to some implementations of the present disclosure, a system includes a sensor, a memory, and a control system. The sensor is configured to generate thoracic impedance data associated with a patient for a period of time. The memory stores machine-readable instructions, and the control system is communicatively coupled to the sensor and the memory. The control system is arranged to provide control signals to one or more electronic devices and including one or more processors configured to execute the machine-readable instructions to: analyze the generated thoracic impedance data associated with the patient; determine, based at least in part on the analysis, respiratory function signals and cardiac function signals, and whether the respiratory function signals and cardiac function signals satisfies a predetermined threshold; and responsive to the determination of the respiratory function signals and cardiac function signals satisfies the predetermined threshold, cause an operation of the one or more electronic devices to be modified.

According to some implementations of the present disclosure, the sensor is communicatively coupled to at least one of the following: a hospital bed side monitor, a health clinic bed side monitor, or an ambulance bedside monitor. The respiratory function signals can include breathing indices on a breath-to-breath basis. The control system is further arranged to provide control signals to one or more electronic devices and including one or more processors configured to execute the machine-readable instructions to analyze a current average and a moving average of the breathing indices, determine, based at least in part on the analysis, a breathing function in real time and whether the determined breathing function satisfies a predetermined threshold, and responsive to determining the breathing function in real time satisfies the predetermined threshold, sending a command to the one or more electronic devices to display a notification to the patient or a caregiver.

According to some implementations of the present disclosure, the cardiac function signals include cardiac indices on a heartbeat-to-heartbeat basis. The control system is further arranged to provide control signals to one or more electronic devices and including one or more processors configured to execute the machine-readable instructions to analyze a current average and a moving average of the cardiac indices, determine, based at least in part on the analysis, a heart function in real time and whether the determined heart function satisfies a predetermined threshold, and responsive to determining the heart function in real time satisfies the predetermined threshold, sending a command to the one or more electronic devices to display a notification to the patient or a caregiver.

According to some implementations of the present disclosure, the sensor is configured as a wearable device communicatively coupled to the patient. The control system is further arranged to provide control signals to one or more electronic devices and including one or more processors configured to execute the machine-readable instructions to generate a medical early warning score (“MEWS”) based at least in part on the determined respiratory function signals and the cardiac function signals.

According to some implementations of the present disclosure, the sensor comprises at least one of a wearable breathing sensor placed on a patient's chest. The control system communicatively coupled to the sensor is within a housing of the wearable breathing sensor. Alternatively, in some implementations, the control system communicatively coupled to the sensor is located remotely from the sensor. In some implementations, the control system is housed in a terminal device. The terminal device can include at least one of a mobile phone, a smart watch, a tablet, a personal computer, or a cloud based computing device. In some implementations of the present disclosure, at least a portion of the respiratory function signals comprises chest wall movement of the patient. In some implementations, at least a portion of the respiratory function signals includes active exhalation time and total exhalation time for each breath of the patient.

According to some implementations of the present disclosure, at least a portion of the respiratory function signals comprises an indication of asthma severity. In some implementations, determining the respiratory function signals satisfies the predetermined threshold signifies that the patient is experiencing abnormal breathing. In some implementations, the thoracic impedance data is generated based on electrocardiogram ECG signals received at the sensor. In some implementations, the control system is further arranged to provide control signals to one or more electronic devices and including one or more processors configured to execute the machine-readable instructions to determine, based at least in part on the analysis, respiratory function signals and cardiac function signals for at least one breathing period, wherein the at least one breathing period is selected from the following: (1) an inspiration, (2) a post-inspiration pause, (3) an exhalation, and (4) a post-expiratory pause. According to some implementations of the present disclosure, the control system is further arranged to provide control signals to one or more electronic devices and including one or more processors configured to execute the machine-readable instructions to determine whether the respiratory function signals and cardiac function signals for the at least one breathing period satisfies a predetermined threshold for the at least one breathing period.

According to some implementations of the present disclosure, a method includes generating, at a sensor, thoracic impedance data associated with a patient for a period of time. The method also includes analyzing the generated thoracic impedance data associated with the patient and determining, based at least in part on the analysis, respiratory function signals and cardiac function signals, and whether the respiratory function signals and cardiac function signals satisfies a predetermined threshold. The method also includes responsive to determining the respiratory function signals and cardiac function signals satisfies the predetermined threshold, causing an operation of one or more electronic devices to be modified.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system for generating thoracic impedance data associated with a user, according to some implementations of the present disclosure;

FIG. 2 is an illustration of a breath breakdown, according to some implementations of the present disclosure;

FIGS. 3A and 3B are an illustration of the respiratory trace and airflow trace relationship, according to some implementations of the present disclosure;

FIG. 4 is an illustration of an identification of a breathing interval, according to some implementations of the present disclosure;

FIG. 5 is an illustration of an electrocardiogram (ECG) trace and a respiratory trace, according to some implementations of the present disclosure;

FIGS. 6A, 6B, 6C and 6D illustrate data generated by the sensor of FIG. 1, according to some implementations of the present disclosure;

FIG. 7 is an illustration of a cardiac trace derived from respiratory waveform scales with stroke volume, according to some implementations of the present disclosure; and

FIG. 8 illustrates a process flow diagram for a method of non-invasive real time monitoring of respiratory and cardiac functions, according to some implementations of the present disclosure.

While the present disclosure is susceptible to various modifications and alternative forms, specific implementations thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION AND EXAMPLE

The present disclosure teaches systems and methods for non-invasive real time monitoring of respiratory and cardiac functions. In addition, the disclosed systems and methods allow for providing a notification to a user or a caregiver upon exceeding critical values. By user, it is meant to include any human person. The user can be a patient in a hospital or any other care facility. Further, the user can be a human living at home in a house, an apartment, a retirement community, a skilled nursing facility, an independent living facility, etc.

The present disclosure teaches non-invasive and real-time assessment of breathing and heart function. The disclosed features are configurable to be used as the basis of a MEWS to assess patients already being monitored with ECG electrodes and bedside monitors in ambulances, clinics, emergency rooms, and in hospitals. In addition, implementations of the present disclosure can be incorporated as a MEWS into a non-invasive wearable device as described in United States Patent Application Publication 2018/0361062A1, filed on Dec. 20, 2018 (incorporated herein by reference).

The present disclosure provides a specific respiratory parameter to measure, and how to measure the specific respiratory parameter. The present disclosure provides a system and method configured to monitor and score any breathing abnormality. In addition, the present disclosure provides a non-invasive cardiac function biomarker that enables detection of declining cardiac output which would alert for possible impending cardiac arrest. Importantly, the breathing and cardiac implementations described herein can be used in combination as the basis for a MEWS for any medical emergency.

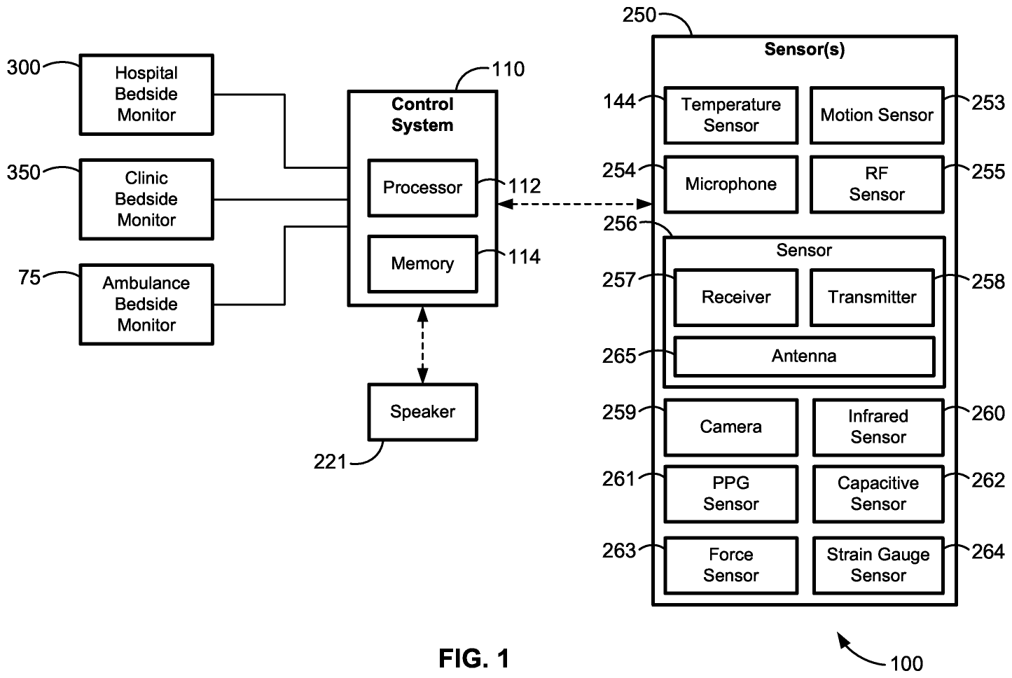

FIG. 1 is a functional block diagram of a system 100 for generating thoracic impedance data associated with a user, according to some implementations of the present disclosure. Referring to FIG. 1, a system 100 includes a control system 110, a hospital bedside monitor 300, a health clinic bedside monitor 350, an ambulance bedside monitor 375, and one or more sensors 250. As described herein, the system 100 generally can be used to frequently monitor data and factors that can be indicative of respiratory and cardiac functions reaching a critical value. While the system 100 is shown as include various elements, the system 100 can include any subset of the elements shown and described herein and/or the system 100 can include one or more additional elements not specifically shown in FIG. 1.

The control system 110 includes one or more processors 112 (hereinafter, processor 112) and a memory device 114. The control system 110 is generally used to control (e.g., actuate) the various components of the system 100 and/or analyze data obtained and/or generated by the components of the system 100. The processor 112 executes machine readable instructions that are stored in the memory device 114 and can be a general or special purpose processor or microprocessor. While one processor 112 is shown in FIG. 1, the control system 110 can include any suitable number of processors (e.g., one processor, two processors, five processors, ten processors, etc.). The memory device 114 can be any suitable computer readable storage device or media, such as, for example, a random or serial access memory device, a hard drive, a solid state drive, a flash memory device, etc. The control system 110 can be coupled to and/or positioned within a housing of the one or more sensor 250, the hospital bedside monitor 300, the health clinic bedside monitor 350, the ambulance bedside monitor 375, or any combination thereof. The control system 110 can be centralized (within one housing) or decentralized (within two or more physically distinct housings).

The one or more sensors 250 include a temperature sensor 252, a motion sensor 253, a microphone 254, a radio-frequency (RF) sensor 255, an impedance sensor 256 (e.g. electrodes also used for ECG), and ECG sensor, a camera 259, an infrared sensor 260, a photoplethysmogram (PPG) sensor 261, a capacitive sensor 262, a force sensor 263, a strain gauge sensor 264, or any combination thereof.

Generally, each of the one or more sensors 250 are configured to output sensor data that is received and stored in the memory device 114 of the control system 110. The impedance sensor 256 includes a receiver 257 and a transmitter 258. The transmitter 258 generates and/or emits waves having a predetermined frequency and/or a predetermined amplitude (e.g., within a high frequency band, within a low frequency band, long wave signals, short wave signals, etc., or any combination thereof). The receiver 257 detects the reflections of the radio waves emitted from the transmitter 258, and this data can be analyzed by the control system 110 to determine a breathing pattern of the user and/or a cardiac signal.

The receiver 257 and/or the transmitter 258 can be wirelessly connected with the control system 110, one or more other devices (e.g., the hospital bedside monitor 300, the health clinic bedside monitor 350, the ambulance bedside monitor 375, etc.). While the based impedance sensor 256 is shown as having a separate receiver and transmitter in FIG. 1, in some implementations, the impedance sensor 256 can include a transceiver that acts as both the receiver 257 and the transmitter 258.

Specifically, the impedance sensor 256 is configured to transmit, receive and measure the breathing intervals in real time and the cardiac signal. This sensor data can be analyzed by one or more processors 112 of the control system 110 to categorize the breathing as normal or abnormal. Any abnormal breathing can be scored (scaled).

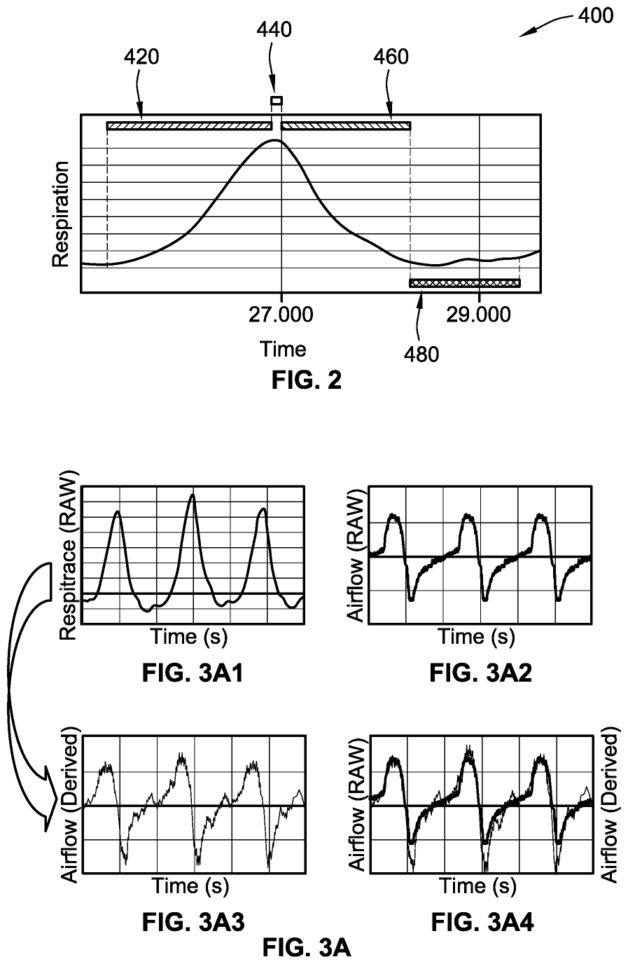

It should be understood that the one or more sensors 250 can include any combination and any number of the sensors described and/or shown herein. The temperature sensor 252 outputs temperature data that can be stored in the memory device 114 of the control system 110 and/or analyzed by the processor 112 of the control system 110. In some implementations, the temperature sensor 252 generates temperatures data indicative of a core body temperature of the resident 20 (FIG. 2), a skin temperature of the user, an ambient temperature, or any combination thereof. The temperature sensor 252 can be, for example, a thermocouple sensor, a thermistor sensor, a silicon band gap temperature sensor or semiconductor-based sensor, a resistance temperature detector, or any combination thereof.

The microphone 254 outputs sound data that can be stored in the memory device 114 of the control system 110 and/or analyzed by the processor 112 of the control system 110. The microphone 254 can be used to record sound(s) related to breathing of the user to determine, for example, to measure the breathing intervals in real time.

A speaker 221 outputs sound waves that are audible to the user. The speaker 221 can be used, for example, as an alarm clock and/or to play an alert or message to the user (e.g., in response to detecting abnormal breathing of the user) and/or to a third party (e.g., a family member of the user, a friend of the user, a caregiver of the user, etc.). In some implementations, the microphone 254 and the speaker 221 can be used collectively used together as a sonar sensor. In such implementations, the speaker 221 generates or emits sound waves at a predetermined interval and the microphone 254 detects the reflections of the emitted sound waves from the speaker 221. The sound waves generated or emitted by the speaker 221 have a frequency that is not audible to the human ear (e.g., below 20 Hz or above around 18 kHz) so as not to disturb the user. Based at least in part on the data from the microphone 254 and the speaker 221, the control system 110 can determine the user's breathing.

The RF sensor 255 generates and/or emits radio waves having a predetermined frequency and/or a predetermined amplitude (e.g., within a high frequency band, within a low frequency band, long wave signals, short wave signals, etc.). The RF sensor 255 also detects the reflections of the radio waves emitted, and this data can be analyzed by the control system 110 to determine the user's breathing.

The camera 259 outputs image data reproducible as one or more images (e.g., still images, video images, thermal images, or a combination thereof) that can be stored in the memory device 114 of the control system 110. The image data from the camera 259 can be used by the control system 110 to determine the user's breathing.

The infrared (IR) sensor 260 outputs infrared image data reproducible as one or more infrared images (e.g., still images, video images, or both) that can be stored in the memory device 114 of the control system 110. The infrared data from the IR sensor 260 can be used to determine the user's breathing. The IR sensor 260 can also be used in conjunction with the camera 259 when measuring movement of the resident 20. The IR sensor 260 can detect infrared light having a wavelength between about 700 nm and about 1 mm, for example, while the camera 259 can detect visible light having a wavelength between about 380 nm and about 740 nm.

The PPG sensor 261 outputs physiological data associated with the user that can be used to determine the user's breathing. The PPG sensor 261 can be worn by the user and/or embedded in clothing and/or fabric that is worn by the user. The physiological data generated by the PPG sensor 261 can be used alone and/or in combination with data from one or more of the other sensors 250 to determine the user's breathing.

The capacitive sensor 262, the force sensor 263, and the strain gauge sensor 264 output data that can be stored in the memory device 114 of the control system 110 and used by the control system 110 individually and/or in combination with data from one or more other sensors 250 to determine a state of the resident 20. In some implementations, the one or more sensors 250 also include a galvanic skin response (GSR) sensor, an electroencephalography (EEG) sensor, an electromyography (EMG) sensor, a blood flow sensor, a respiration sensor, a pulse sensor, a sphygmomanometer sensor, an oximetry sensor, an oxygen sensor, or any combination thereof.

While shown separately in FIG. 1, the one or more sensors 250 can be integrated in and/or coupled to any of the components of the system 100 including the control system 110, the external devices (e.g., the configurable bed apparatus 350, the footwear garment 400), or any combination thereof. For example, the microphone 254 and the speaker 221 can be integrated in and/or coupled to the control system 110, the hospital bedside monitor 300, the health clinic bedside monitor 350, the ambulance bedside monitor 375, or a combination thereof. In some implementations, at least one of the one or more sensors 250 are not coupled to the control system 110, or the external devices, and is positioned generally adjacent to the user (e.g., coupled to or positioned on a nightstand, coupled to a mattress, coupled to a ceiling, coupled to a wall, coupled to a lighting device, etc.).

FIG. 2 is an illustration of a breath breakdown 400, according to some implementations of the present disclosure. Breathing intervals of an individual breath can be quantified using data obtained by the sensor 250 (of FIG. 1). For example, the breathing intervals can be quantified from impedance based respiratory waveforms. In other embodiments, the breathing intervals can be quantified from any types of breathing trace, henceforth referred to as breathing waveforms. The quantifying of the breathing intervals enables the present disclosure to identify and quantify breathing abnormalities in real time.

As illustrated in FIG. 2, the breath breakdown 400 represents a breathing period (i.e. the duration of a complete breath). The breath breakdown 400 can be divided into four physiologically distinct intervals: (i) inspiration 420, (ii) post-inspiratory pause 440, (iii) exhalation 460 and (iv) post-expiratory pause 480. Normal and abnormal breathing patterns can be identified and scored by measuring these four breathing intervals. Implementations of the present disclosure provide an ability to identify the start and stop times of each interval to be used to identify and score any breathing condition in real-time, whether normal or abnormal (including anaphylaxis, asthma, COPD, heart failure, etc.). Identification of the specific patterns of these intervals during breathing can be used to categorize and score breathing into any breathing pathology.

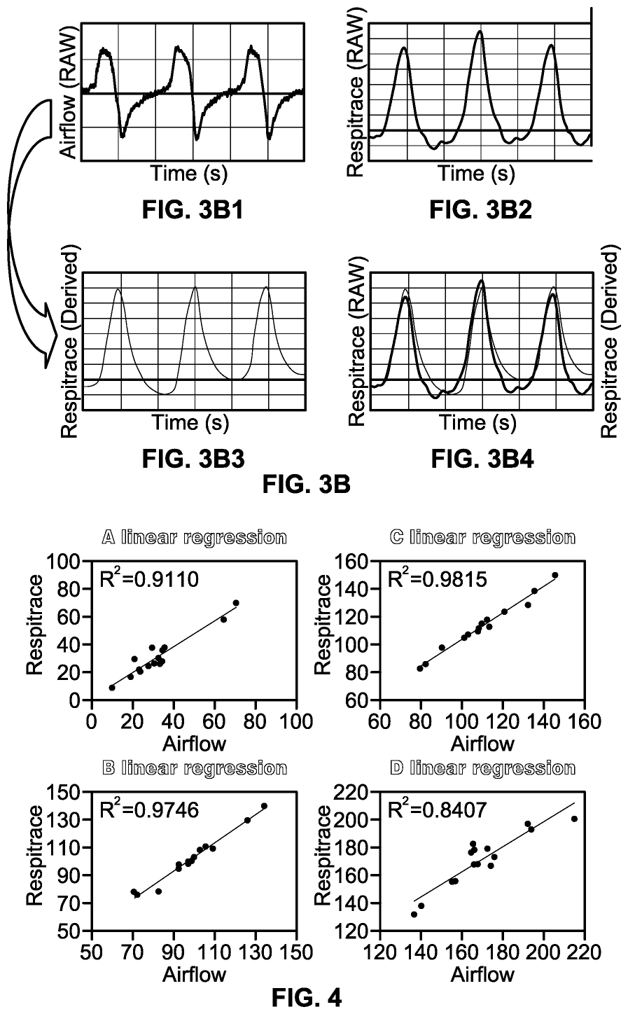

FIGS. 3A and 3B are an illustration of the respiratory trace and airflow trace relationship, according to some implementations of the present disclosure. As illustrated in FIG. 3, three datasets (ECG, respiratory waveform, and airflow) associated with a user were simultaneously recorded during normal breathing and during apnea (20-second breath holds). Respiratory waveform data was obtained using a bedside monitor and ECG leads (e.g. electrodes) generally accepted in the state of the art. Airflow data were obtained using an airflow meter.

FIG. 3A illustrates the transformation of the raw respiratory trace (top left 3A1) to an artificial airflow trace (bottom left 3A3). Furthermore, the transformed signal is then compared to the measured airflow trace (top right 3A2). Both the measured airflow and the artificial (transformed) airflow traces are plotted together (bottom right 3A4).

FIG. 3B illustrates the transformation of the raw airflow trace (top left 3B1) to an artificial respiratory trace (bottom left 3B3) is illustrated. The transformed signal is then compared to the measured respiratory trace (top right 3B2). Both measured respiratory and artificial (transformed) respiratory traces are plotted together (bottom right 3B4). As illustrated herein, the airflow waveforms are generated by transforming simultaneously recorded respiratory waveform data (FIG. 3A) and conversely that respiratory waveforms could be generated by transforming simultaneously recorded airflow data (FIG. 3B). As a result, the present disclosure allows for validation of the measured breathing intervals from respiratory waveforms against gold standard airflow meters. To validate the measured breathing intervals, breathing intervals are quantified from airflow data. The breathing intervals are analyzed and compared to airflow data and derived airflow from simultaneously recorded respiratory waveform data.

FIGS. 4A-4D illustrates graphs showing an identification of a breathing interval, according to some implementations of the present disclosure. As illustrated herein, start times from breathing traces are nearly identical to those determined using simultaneously recorded airflow data. Shown are breathing interval start times determined from respiratory waveform data as a function of those determined from Airflow data. p<0.0001, n=15 for all interval start times. Specifically, FIGS. 4A-4D illustrates nearly identical time-points for the start of the four breathing intervals (i.e. start inspiration (A), start end-inspiratory pause (B), start expiration (C), and start end-expiratory pause (D)) from both datasets. These results illustrate that breathing intervals can be similarly identified using respiratory waveforms or gold standard airflow. As a result, respiratory waveforms can be reliably used to non-invasively monitor and score breathing in real-time. In some implementations, ECG impedance-based respiratory waveforms are validated as a surrogate of airflow. Therefore, this validation enables hospitals and medical professionals worldwide to non-invasively monitor and score breathing in real time.

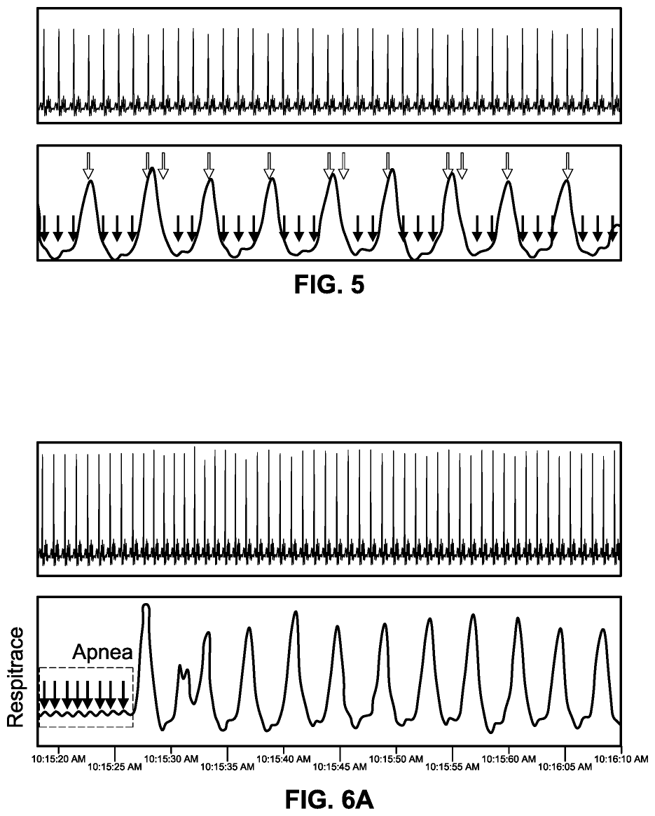

FIG. 5 is an illustration of an ECG trace (top) and a respiratory trace (bottom), according to some implementations of the present disclosure. The effect of each heartbeat on respiratory waveform 501 is more pronounced during pauses in breathing 502, and subtler but noticeable throughout the rest of the respiratory cycle 503.

Based on the provided traces, an index of cardiac function can be derived through transformation of the respiratory waveform. The index of cardiac function also scales with cardiac output. The identification of the index is advantageous, as is the identification of a non-invasive marker that can be used to monitor the cardiac output. In some implementations, the signal can be non-invasively acquired and quantified in real time and monitored. In this way, a practitioner, or the user themselves are able to identify declining cardiac function as a warning of impending cardiac arrest or need for urgent medical attention and resuscitation. In some implementations, the system 100 (FIG. 1) can be utilized to support care and emergency monitoring for patients connected to bedside monitors through electrode leads in ambulances, medical offices, clinics, emergency rooms or in hospitals. Alternatively, the system 100 (FIG. 1) can be utilized as part of a wearable technology to monitor for medical emergencies by anybody in the community. In addition, the system 100 (FIG. 1) can be utilized for non-invasive monitoring of cardiac output during medical rehabilitation of patients or of athletes during training.

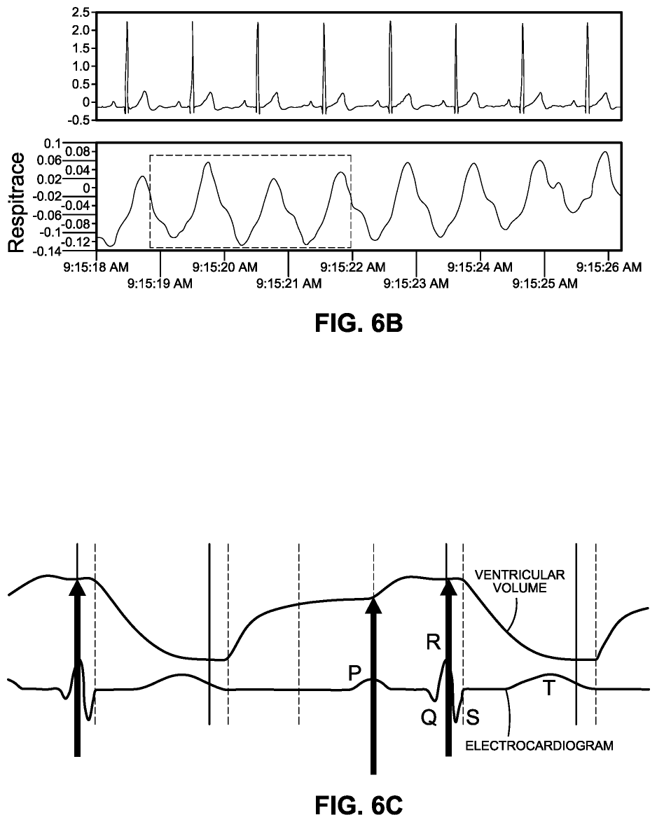

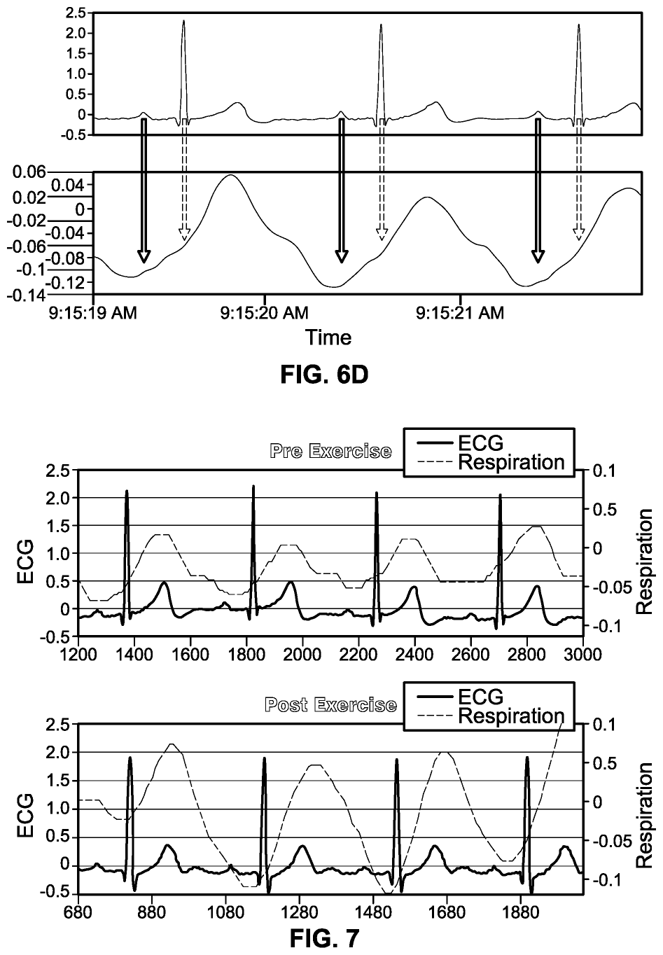

FIGS. 6A, 6B, 6C and 6D illustrate data generated by the sensor 250 (of FIG. 1), according to some implementations of the present disclosure. As illustrated herein, it is determined that each heartbeat imprinted the respiratory waveform, and that this effect was more pronounced during pauses in breathing or when breathing motion slowed (i.e. near or during end-inspiratory and end-expiratory pauses (see FIG. 5). The timing of the heartbeat with respect to breathing effects the respiratory waveform, specifically, by changing the shape of the waveform. As a result, it becomes difficult during an analysis to precisely identify some transitions from one breathing interval to the next.

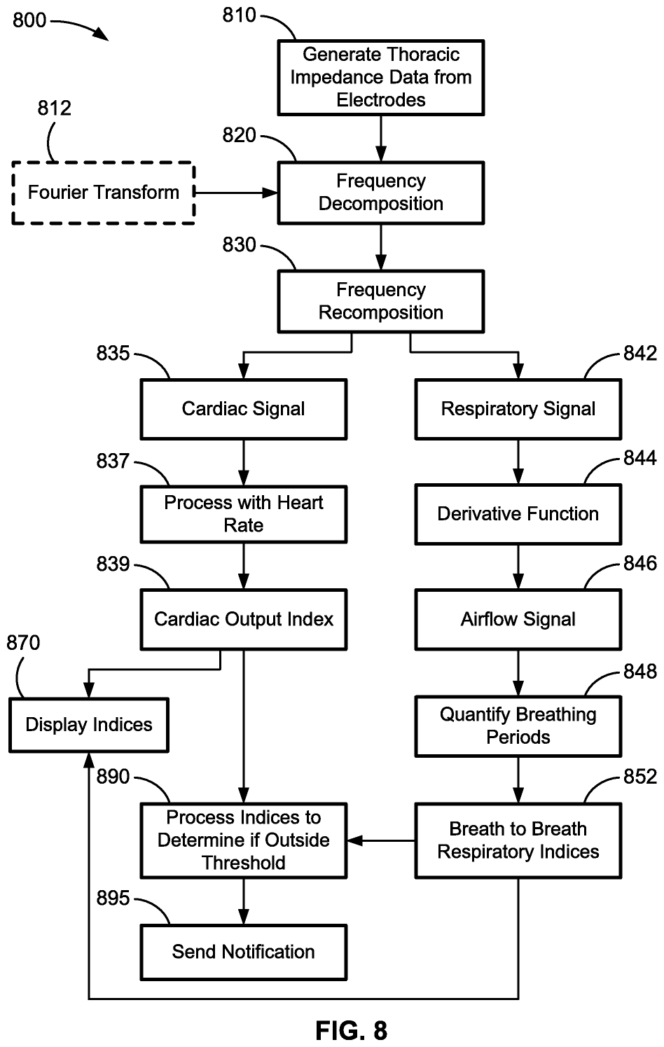

It was determined that mathematical subtraction of the cardiac signal from the respiratory waveform would significantly enhance the accuracy of the breathing analysis. In addition, mathematical isolating the heart beat signal from the respiratory waveform outputs an index of cardiac function as described below with respect to FIG. 8. To more clearly characterize the heartbeat effect on the respiratory waveform, the ECG and respiratory waveform data are simultaneously captured and analyzed during normal breathing that followed 20 seconds of voluntary apnea (FIG. 6A). Note that the respiratory waveform during prolonged apnea undulates with identical frequency to the heartbeat (timing of heartbeats during apnea denoted by white arrows as determined from the ECG at the top of FIG. 6A).

FIG. 6B illustrates a magnification of the respiratory waveform from the inset in FIG. 6A with a simultaneously recorded ECG. Close inspection of heartbeat dependent undulations such as those shown in FIG. 6B revealed that the undulations inversely emulate a typical left ventricle stroke volume (i.e. how much blood volume the heart pumps per beat) waveform (FIG. 6C) that can only be generated using cardiac ultrasound. Furthermore, the timing and effects of intra-cardiac events such as atrial (arrow) and ventricular (dashed arrow) contractions can be similarly identified in the left ventricle volume trace (FIG. 6C) and in the apnea portion of the Respiratory trace (see FIG. 6D, a magnification of the inset in FIG. 6B).

As a result, the heartbeat dependent undulations in the respiratory waveform trace can be used as a non-invasive biomarker of cardiac function. The magnitude of cardiac undulations seen on respiratory traces (discussed below with respect to FIG. 7 during periods of voluntary apnea) significantly increased following normal exercise. Thus, the cardiac ‘signal’ identified herein indeed scales with cardiac function. This finding implied that if this heartbeat signal could be both isolated and removed from the respiratory waveform signal, then the breathing interval transition time points can be more accurately identified. Furthermore, an index signal of cardiac function (i.e. surrogate of stroke volume and of cardiac output) can be separately generated.

FIG. 7 is an illustration of a cardiac trace derived from respiratory waveform scales with stroke volume, according to some implementations of the present disclosure. In order to isolate and remove the heartbeat signal from the respiratory waveform, a Fourier Transformation may be applied to split the waveform into multiple components based on frequency and then an inverse Fourier Transformation may be applied to separately reconstruct (i) the heartbeat signal and (ii) the respiratory signal with the heartbeat signal removed. The significant similarity between the heartbeat signal seen during apnea (FIGS. 6B, 6D and FIG. 7) and the heartbeat signal derived from the respiratory waveform using the Fourier and inverse Fourier transformations.

The newly derived respiratory waveform signal no longer contains the heartbeat ‘noise’. As a general note, ‘cleaning’ the respiratory waveform data improves the sensitivity and specificity of the breathing analysis described herein. Quantification of the heart beat signal provides a non-invasive index of cardiac function. In addition to the disclosed validation, changes in cardiac output from respiratory waveforms can also be measured. The signal from the sensor 250 (of FIG. 1) can be used to non-invasively monitor cardiac output and would identify deterioration in cardiac output necessary to detect medical emergencies prior to cardiac arrest.

One advantage of the disclosure is that because ECG systems are ubiquitously used in ambulances, clinics, emergency rooms and hospitals throughout the world, our invention enables hospitals and medical professionals worldwide to non-invasively monitor cardiac function with existing and ubiquitously used hardware to identify medical emergencies. The technology would also provide the means for a wearable device to detect impending respiratory and cardiac failure that precede cardiac arrest.

### Example Methods for Monitoring of Respiratory and Cardiac Function

FIG. 8 illustrates a flow chart showing an example method 800 for non-invasive real time monitoring of respiratory and cardiac function. One or more of the steps of the method 800 described herein can be implemented using the system 100 (FIG. 1) or other suitable systems. First, a sensor measures and generates thoracic impedance data 810 for a period of time. As discussed herein, the sensor can include an ECG based impedance sensor 256 that may include electrodes. The ECG based impedance sensor 256 may include two or more electrodes, and a processing system for measuring various impedances between the electrodes and outputting data relating to the impedance signal.

As previously discussed, the sensor is communicatively coupled to at least one of the following: a hospital bed side monitor, a health clinic bed side monitor, or an ambulance bedside monitor. Alternatively, the sensor can be configured as a wearable device communicatively coupled to the patient. Alternatively, the sensor can be one of many wearable breathing sensors placed on a patient's chest.

Next, the method 800 includes analyzing, using a control system, the generated thoracic impedance data associated with the patient using various functions to split the signal into a cardiac signal 835 and a respiratory signal 842. For instance, the system may apply a frequency decomposition 820 function to separate the frequency components of the signal. For instance, a Fourier transform or other suitable functions may be utilized to separate out the frequencies of the impedance data. In some examples, various frequency components will then be filtered out that are related to noise.

Then, the frequency spectrum can be recomposed 830 into a cardiac signal 835 and a respiratory signal 842. In some examples, the respiratory signal 842 frequency is much lower than the cardiac signal 835 frequency and is thus easily and cleanly separated once the impedance signal is initially decomposed into its frequencies. For instance, various frequency filters could be used to separate the signals (and remove noise) and then a frequency recomposition 830 function (e.g., inverse Fourier transform) could be applied to reconstruct the cardiac signal 835 and separately reconstruct the respiratory signal 842.

### Cardiac Signal Processing

Next the cardiac signal 835 may be further processed to output various indices and other cardiac function related measures. For instance, the cardiac signal may be processed with the heart rate 837. In one example, it may be multiplied by the heart rate to produce a surrogate stroke volume index trace. Based on this function or model, the system could then output a volume index of each heart beat and a cardiac output index that incorporate beat frequency.

After processing these indices, the system may first display the indices 870 on the various monitors or other displays disclosed herein. In other examples, these metrics may be processed to determine if they are outside a threshold 890, or a trend indicates they will be outside a threshold 890 that indicates an imminent or approaching health problem. Then in some instances, this system may automatically send a notification 895 to the patient or caregiver through various modes including text messages, notifications, emails, calls, etc.

### Respiratory Signal Processing

Additionally, the respiratory signal 842 may be further processed after separation as described above to output various indices and other respiratory function related measures. For instance, a derivative function 844 may be applied to the respiratory signal 842 to output a derived respiratory airflow waveform 846. For example, a function applying the derivative of the respiratory signal 842 will output a surrogate airflow trace. Next, a function or model can be applied to quantify the breathing periods 848 between key physiological transition points within breathing cycles, including by identifying points with a defined set of parameters of the maximum and minimum derived airflow values.

The respiratory indices may be output on a breath-to-breath basis. In such implementations, a current average and a moving average of the breathing indices can be analyzed. In some implementations, at least a portion of the respiratory function signals includes an indication of asthma severity. In some examples, the system will cause an operation of one or more electronic devices to be modified in response to determining the respiratory function signals and/or cardiac function signals satisfies a predetermined threshold. Determining that the respiratory function signals satisfies the predetermined threshold may signify that the patient is experiencing abnormal or path breathing.

After processing these indices, the system may first display the indices 870 on the various monitors or other displays disclosed herein. In other examples, these metrics may be processed to determine if they are outside a threshold 890, or a trend indicates they will be outside a threshold 890 that indicates an imminent or approaching health problem. Then in some instances, this system may automatically send a notification 895, to the patient or caregiver through various modes including text messages, notifications, emails, calls, etc.

While the present disclosure has been described with reference to one or more particular implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The present invention can be defined in any of the following numbered paragraphs:
1. A system comprising:
   a sensor configured to generate thoracic impedance data associated with a patient for a period of time;
   a memory storing machine-readable instructions; and
   a control system communicatively coupled to the sensor and arranged to provide control signals to one or more electronic devices and including one or more processors configured to execute the machine-readable instructions to:
   analyze the generated thoracic impedance data associated with the patient;
   determine, based at least in part on the analysis, respiratory function signals and cardiac function signals, and whether the respiratory function signals and cardiac function signals satisfies a predetermined threshold; and responsive to the determination of the respiratory function signals and cardiac function signals satisfies the predetermined threshold, cause an operation of the one or more electronic devices to be modified.
2. The system of paragraph 1, wherein the sensor is communicatively coupled to at least one of the following: a hospital bed side monitor, a health clinic bed side monitor, or an ambulance bedside monitor.
3. The system of any one of paragraphs 1-2, wherein the respiratory function signals comprise breathing indices on a breath-to-breath basis.
4. The system of paragraph 3, wherein the control system is further arranged to provide control signals to one or more electronic devices and including one or more processors configured to execute the machine-readable instructions to: analyze a current average and a moving average of the breathing indices;
   determine, based at least in part on the analysis, a breathing function in real time and whether the determined breathing function satisfies a predetermined threshold; and
   responsive to the determination of the breathing function in real time satisfies a predetermined threshold, sending a command to the one or more electronic devices to display a notification to the patient or a caregiver.
5. The system of any one of paragraphs 1-4, wherein the cardiac function signals comprise cardiac indices on a heartbeat-to-heartbeat basis.
6. The system of paragraph 5, wherein the control system is further arranged to provide control signals to one or more electronic devices and including one or more processors configured to execute the machine-readable instructions to: analyze a current average and a moving average of the cardiac indices;
   determine, based at least in part on the analysis, a heart function in real time and whether the determined heart function satisfies a predetermined threshold; and responsive to the determination of the heart function in real time satisfies a predetermined threshold, sending a command to the one or more electronic devices to display a notification to the patient or a caregiver.
7. The system of any one of paragraphs 1-6, wherein the sensor is configured as a wearable device communicatively coupled to the patient.
8. The system of any one of paragraphs 1-7, wherein the control system is further arranged to provide control signals to one or more electronic devices and including one or more processors configured to execute the machine-readable instructions to:
   generate a medical early warning score (MEWS) based at least in part on the determined respiratory function signals and the cardiac function signals.
9. The system of any one of paragraphs 1-8, wherein the sensor comprises at least one of a wearable breathing sensor placed on a patient's chest.
10. The system of paragraph 9, wherein the control system communicatively coupled to the sensor is within a housing of the wearable breathing sensor.
11. The system of paragraph 9, wherein the control system communicatively coupled to the sensor is located remotely from the sensor.
12. The system of any one of paragraphs 1-11, wherein the control system is housed in a terminal device, wherein the terminal device comprises at least one of a mobile phone, a smart watch, a tablet, a personal computer, a cloud based computing device.
13. The system of any one of paragraphs 1-12, wherein at least a portion of the respiratory function signals comprises chest wall movement of the patient.
14. The system of any one of paragraphs 1-13, wherein at least a portion of the respiratory function signals comprises active exhalation time and total exhalation time for each breath of the patient.
15. The system of any one of paragraphs 1-14, wherein at least a portion of the respiratory function signals comprises an indication of asthma severity.
16. The system of any one of paragraphs 1-15, wherein determining the respiratory function signals satisfies the predetermined threshold signifies that the patient is experiencing abnormal breathing.
17. The system of any one of paragraphs 1-16, wherein the thoracic impedance data is generated based on electrocardiogram ECG signals received at the sensor.
18. The system of any one of paragraphs 1-17, wherein the control system is further
   arranged to provide control signals to one or more electronic devices and including one or more processors configured to execute the machine-readable instructions to:
   determine, based at least in part on the analysis, respiratory function signals and cardiac function signals for at least one breathing period,
   wherein the at least one breathing period is selected from the following: (1) an inspiration, (2) a post-inspiration pause, (3) an exhalation, and (4) a post-expiratory pause.
19. The system of paragraph 17, wherein the control system is further arranged to provide control signals to one or more electronic devices and including one or more processors configured to execute the machine-readable instructions to:
   responsive to the determination of the respiratory function signals and cardiac function signals for the at least one breathing period, determine whether the respiratory function signals and cardiac function signals for the at least one breathing period satisfies a predetermined threshold for the at least one breathing period.
20. A method comprising:
   generating, at a sensor, thoracic impedance data associated with a patient for a period of time;
   analyzing the generated thoracic impedance data associated with the patient;
   determining, by a control system, based at least in part on the analysis, respiratory function signals and cardiac function signals, and whether the respiratory function signals and cardiac function signals satisfies a predetermined threshold; and
   responsive to determining the respiratory function signals and cardiac function signals satisfies the predetermined threshold, causing an operation of one or more electronic devices to be modified.
21. The method of paragraph 20, wherein the sensor is communicatively coupled to at least one of the following: a hospital bed side monitor, a health clinic bed side monitor, or an ambulance bedside monitor.
22. The method of any one of paragraphs 20-21, wherein the respiratory function signals comprise breathing indices on a breath-to-breath basis.
23. The method of any one of paragraphs 20-22, further comprising:
   analyzing a current average and a moving average of the breathing indices;
   determining, based at least in part on the analysis, a breathing function in real time and whether the determined breathing function satisfies a predetermined threshold; and
   responsive to the determination of the breathing function in real time satisfies a predetermined threshold, sending a command to the one or more electronic devices to display a notification to the patient or a caregiver.
24. The method of any one of paragraphs 20-23, wherein the cardiac function signals comprise cardiac indices on a heartbeat-to-heartbeat basis.
25. The method of paragraph 24, further comprising:
   analyzing a current average and a moving average of the cardiac indices;
   determining, based at least in part on the analysis, a heart function in real time and whether the determined heart function satisfies a predetermined threshold; and
   responsive to determining the heart function in real time satisfies a predetermined threshold, sending a command to the one or more electronic devices to display a notification to the patient or a caregiver.
26. The method of any one of paragraphs 20-25, wherein the sensor is configured as a wearable device communicatively coupled to the patient.
27. The method of any one of paragraphs 20-26, further comprising:
   generating a medical early warning score (MEWS) based at least in part on the determined respiratory function signals and the cardiac function signals.
28. The method of any one of paragraphs 20-27, wherein the sensor comprises at least one of a wearable breathing sensor placed on a patient's chest.
29. The method of any one of paragraphs 20-28, wherein the control system communicatively coupled to the sensor is within a housing of the wearable breathing sensor.
30. The method of any one of paragraphs 20-29, wherein the control system communicatively coupled to the sensor is located remotely from the sensor.
31. The method of any one of paragraphs 20-30, wherein the control system is housed in a terminal device, wherein the terminal device comprises at least one of a mobile phone, a smart watch, a tablet, a personal computer, or a cloud based computing device.
32. The method of any one of paragraphs 20-31, wherein at least a portion of the respiratory function signals comprises chest wall movement of the patient.
33. The method of any one of paragraphs 20-32, wherein at least a portion of the respiratory function signals comprises active exhalation time and total exhalation time for each breath of the patient.
34. The method of any one of paragraphs 20-33, wherein at least a portion of the respiratory function signals comprises an indication of asthma severity.
35. The method of any one of paragraphs 20-34, wherein determining the respiratory function signals satisfies the predetermined threshold signifies that the patient is experiencing abnormal breathing.
36. The method of any one of paragraphs 20-35, wherein the thoracic impedance data is generated based on impedance data detected between electrodes.
37. The method of any one of paragraphs 20-36, further comprising:
   determining, based at least in part on the analysis, respiratory function signals and cardiac function signals for at least one breathing period,
   wherein the at least one breathing period is selected from the following: (1) an inspiration, (2) a post-inspiration pause, (3) an exhalation, and (4) a post-expiratory pause.
38. The method of paragraph 37, further comprising determining whether the respiratory function signals and cardiac function signals for the at least one breathing period satisfies a predetermined threshold for the at least one breathing period.
39. A system comprising:
   a sensor configured to generate thoracic impedance data associated with a patient for a period of time;
   a memory storing machine-readable instructions; and
   a control system communicatively coupled to the sensor and arranged to provide control signals to one or more electronic devices and including one or more processors configured to execute the machine-readable instructions to:
   analyze the generated thoracic impedance data with a first model to output a respiratory signal and a cardiac signal; and
   display the respiratory signal and the cardiac signal.
40. The system of paragraph 39, wherein the first model comprises a frequency decomposition function, a filter, and a frequency recomposition function.
41. The system of any one of paragraphs 39-40, wherein the control system is further configured to process the cardiac signal with a second model to output a cardiac index.
42. The system of paragraph 41, wherein the second model comprises multiplication with a heart rate.
43. The system of any one of paragraphs 39-42, wherein the control system is further configured to process the respiratory signal with a third model to output a respiratory index.
44. The system of paragraph 43, wherein the third model comprises a derivative function and a quantification of breathing periods.
45. The system of paragraph 39, wherein the control system is further configured to process the cardiac index and the respiratory index to output an indication of whether the patient has an imminent medical emergency.
46. The system of paragraph 43, wherein the signal is a notification sent to a mobile device.

1. A system comprising:
a sensor configured to generate thoracic impedance data associated with a patient for a period of time;
a memory storing machine-readable instructions; and
a control system communicatively coupled to the sensor and arranged to provide control signals to one or more electronic devices and including one or more processors configured to execute the machine-readable instructions to:
analyze the generated thoracic impedance data associated with the patient;
determine, based at least in part on the analysis, respiratory function signals and cardiac function signals, and whether the respiratory function signals and cardiac function signals satisfies a predetermined threshold; and
responsive to the determination of the respiratory function signals and cardiac function signals satisfies the predetermined threshold, cause an operation of the one or more electronic devices to be modified.

2. The system of claim 1, wherein the sensor comprises at least one wearable breathing sensor placed on the patient's chest communicatively coupled to the patient, or is communicatively coupled to at least one of the following: a hospital bed side monitor, a health clinic bed side monitor, or an ambulance bedside monitor.

3. The system of claim 1, wherein the respiratory function signals comprise breathing indices on a breath-to-breath basis.

4. The system of claim 1, wherein the control system is further arranged to provide control signals to one or more electronic devices and including one or more processors configured to execute the machine-readable instructions to:
analyze a current average and a moving average of the breathing indices;
determine, based at least in part on the analysis, a breathing function in real time and whether the determined breathing function satisfies a predetermined threshold; and
responsive to the determination of the breathing function in real time satisfies a predetermined threshold, sending a command to the one or more electronic devices to display a notification to the patient or a caregiver.

5. The system of claim 1, wherein the cardiac function signals comprise cardiac indices on a heartbeat-to-heartbeat basis.

6. The system of claim 5, wherein the control system is further arranged to provide control signals to one or more electronic devices and including one or more processors configured to execute the machine-readable instructions to:
analyze a current average and a moving average of the cardiac indices;
determine, based at least in part on the analysis, a heart function in real time and whether the determined heart function satisfies a predetermined threshold; and
responsive to the determination of the heart function in real time satisfies a predetermined threshold, sending a command to the one or more electronic devices to display a notification to the patient or a caregiver.

7. (canceled)

8. The system of claim 1, wherein the control system is further arranged to provide control signals to one or more electronic devices and including one or more processors configured to execute the machine-readable instructions to:
generate a medical early warning score (MEWS) based at least in part on the determined respiratory function signals and the cardiac function signals.

9. (canceled)

10. The system of claim 1, wherein the control system communicatively coupled to the sensor is within a housing of the wearable breathing sensor or located remotely from the sensor.

11. (canceled)

12. The system of claim 1, wherein the control system is housed in a terminal device, wherein the terminal device comprises at least one of a mobile phone, a smart watch, a tablet, a personal computer, a cloud based computing device.

13. The system of claim 1, wherein at least a portion of the respiratory function signals comprises at least one of the group of: chest wall movement of the patient, active exhalation time and total exhalation time for each breath of the patient, or an indication of asthma severity.

14.-15. (canceled)

16. The system of claim 1, wherein determining the respiratory function signals satisfies the predetermined threshold signifies that the patient is experiencing abnormal breathing.

17. The system of claim 1, wherein the thoracic impedance data is generated based on electrocardiogram ECG signals received at the sensor.

18. The system of claim 1, wherein the control system is further arranged to provide control signals to one or more electronic devices and including one or more processors configured to execute the machine-readable instructions to:
determine, based at least in part on the analysis, respiratory function signals and cardiac function signals for at least one breathing period,
wherein the at least one breathing period is selected from the following: (1) an inspiration, (2) a post-inspiration pause, (3) an exhalation, and (4) a post-expiratory pause.

19. The system of claim 18, wherein the control system is further arranged to provide control signals to one or more electronic devices and including one or more processors configured to execute the machine-readable instructions to:
responsive to the determination of the respiratory function signals and cardiac function signals for the at least one breathing period, determine whether the respiratory function signals and cardiac function signals for the at least one breathing period satisfies a predetermined threshold for the at least one breathing period.

20. A method comprising:
generating, at a sensor, thoracic impedance data associated with a patient for a period of time;
analyzing the generated thoracic impedance data associated with the patient;
determining, by a control system, based at least in part on the analysis, respiratory function signals and cardiac function signals, and whether the respiratory function signals and cardiac function signals satisfies a predetermined threshold; and
responsive to determining the respiratory function signals and cardiac function signals satisfies the predetermined threshold, causing an operation of one or more electronic devices to be modified.

21.-38. (canceled)

39. A system comprising:
a sensor configured to generate thoracic impedance data associated with a patient for a period of time;
a memory storing machine-readable instructions; and
a control system communicatively coupled to the sensor and arranged to provide control signals to one or more electronic devices and including one or more processors configured to execute the machine-readable instructions to:
analyze the generated thoracic impedance data with a first model to output a respiratory signal and a cardiac signal; and
display the respiratory signal and the cardiac signal.

40. The system of claim 39, wherein the first model comprises a frequency decomposition function, a filter, and a frequency recomposition function.

41. The system of claim 39, wherein the control system is further configured to process the cardiac signal with a second model to output a cardiac index, wherein the second model comprises multiplication with a heart rate.

42. (canceled)

43. The system of claim 39, wherein the control system is further configured to process the respiratory signal with a third model to output a respiratory index, wherein the third model comprises a derivative function and a quantification of breathing periods, or wherein the respiratory signal is a notification sent to a mobile device.

44. (canceled)

45. The system of claim 39, wherein the control system is further configured to process the cardiac index and the respiratory index to output an indication of whether the patient has an imminent medical emergency.

46. (canceled)
